(19) United States
(12) Patent Application Publication
(10) Pub. No.: US 2023/0046819 A1
(43) Pub. Date: Feb. 16, 2023

(54) COMPOSITE FINDER SYSTEM AND METHOD OF USE THEREOF

(71) Applicant: Andrew Janiga, Nashua, NH (US)

(72) Inventor: Andrew Janiga, Nashua, NH (US)

(21) Appl. No.: 17/403,664

(22) Filed: Aug. 16, 2021

(51) Int. Cl. A61C 19/04

(52) U.S. Cl. CPC A61C 19/04

(57) ABSTRACT

Provided is a method to detect dental composite materials by using a dental tool that provides a source of ultraviolet light. The method includes steps of projecting the source of ultraviolet light to be directed onto one or more dental composite materials on a tooth of a patient. The dental tools used for the method include a visible light source, an ultraviolet light source and a switch which switches light projected from the tool between visible light and ultraviolet light.

### CROSS REFERENCE TO RELATED APPLICATIONS

This application claims priority to and benefit of U.S. Provisional Patent Application No. 63/093,184, filed on Oct. 17, 2020, which is hereby incorporated by reference herein in its entirety.

### FIELD OF THE INVENTION

The disclosure provides a method of using a composite finder system for visualizing dental composites. The method includes projecting ultraviolet light with a wavelength ranging 100-400 nm and detecting fluorescence of the dental composite materials for visual detection.

### BACKGROUND

Dental composites, commonly referred to as “dental fillings” or “tooth-colored fillings” are used widely in dentistry. Advances in the science of dental composites has led to their color being indistinguishable from natural teeth. While there exists application and manufacturing techniques for dental composites used in treatment of dental caries, dental composite detection and/or removal due to various reasons remain problematic. Old dental composites are removed when new cavities form underneath them and when they crack from general tooth wear or trauma. In addition, clear aligner products (e.g., Invisalign) utilize dental composites on teeth to help make proper orthodontics movements. When treatment is finished, these dental composites need to be removed.

To date, the uses of fluorescent light sources in dentistry that aid in identifying dental composite materials are not reported. However, there are some methods and apparatuses that utilize fluorescent for identifying dental decay. For example, U.S. Pat. No. 4,290,433 to Alfano describes a method to detect dental caries by comparing the excited luminescence in two different wavelengths. U.S. Pat. Application 2005/0003323 to Katsuda describes a hand-held imaging apparatus suitable for medical or dental applications, using fluorescence imaging. U.S. Pat. No. 8,447,087 to Wong also describes an apparatus and method for dental imaging that detects dental caries using fluorescence and scattering. Fluorescence, by definition, is the absorption of light by a substance and its emission at the same time at a usually longer wavelength. Such a substance emits more visible light than it receives, making it appear brighter than a non-fluorescent substance which, at best, can only reflect the visible light that is falling on it. While the disclosed methods and apparatus show imaging methods for dental caries detection, none of the cited references employ a fluorescence imaging device that detects dental composite materials on treated dental caries based on the metamerism.

Thus, there is a need in the art for an improved non-invasive and efficient imaging method and a system for dental composite material detection.

### SUMMARY OF THE INVENTION

An object of the invention is to provide a method for assessing human teeth by using a dental tool having at least one mounted light source to detect fluorescence of dental composites on one or more teeth of the human subject.

One aspect of the disclosure provides a method for assessing human teeth which comprises the steps of projecting an ultraviolet light on the teeth of a human subject and detecting fluorescence of dental composites on one or more teeth of the human subject. The method may further comprise a step of recording a tooth location for each of the one or more teeth where fluorescence of dental composites was detected. In some embodiments, the projecting is performed with a pen light. In other embodiments, the projecting is performed with a dental overhead light or a light mounted on dental loupes. In some embodiments, the projecting is performed with a light attached to eyeglasses or loupes. In some embodiments, the projecting is performed with a dental microscope operating light. In some embodiments, the ultraviolet light has a spectrum of wavelengths ranging from 100 nm to 400 nm. The method may further include using a camera to perform the detecting step and a computer to perform the recording step.

Another aspect of the disclosure provides a dental inspection tool which comprises a visible light source, an ultraviolet light source, and a switch which switches light projected from the tool between visible light and ultraviolet light. In some embodiments, the tool may be configured as a dental overhead light, a stand lamp, dental loupes, a handheld device, a penlight or a dental microscope. The tool may further comprise a disposable cover while the body of the tool is reusable. In some embodiments, the tool may have a controller to set the light source to emit a specified range of wavelengths.

Additional features and advantages of the present invention will be set forth in the description of disclosure that follows, and in part will be apparent from the description of may be learned by practice of the disclosure. The disclosure will be realized and attained by the compositions and methods particularly pointed out in the written description and claims hereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a perspective view of the UV light source configured in a shape of an exemplary dental pen light.

FIG. 2 is a perspective view of the UV light source mounted on a pair of telescopic binoculars affixed to a pair of eyeglasses to form dental loupes.

FIG. 3 is a perspective view of the UV light source mounted on an exemplary dental microscopy device.

FIG. 4 is a perspective view of the UV light source mounted on an exemplary dental overhead light system.

FIGS. 5A-B show representative images of teeth with dental composite material viewed under natural light (A) and under UV light (B) at wavelength of 390 nm.

### DETAILED DESCRIPTION

The preferred embodiments of the present disclosure are directed toward methods of detecting dental composites on at least one tooth of a subject by using a dental tool providing proper lighting for detection. In particular, the preferred embodiments are directed toward a method of detecting dental composites by using a dental tool which is capable of projecting ultraviolet light onto the tooth of the subject and detecting fluorescence of dental composites upon ultraviolet light exposure without adding or applying additional fluorophores.

One aspect of the disclosure provides a method for assessing human teeth which comprises the steps of projecting an ultraviolet light on the teeth of a human subject and detecting fluorescence of dental composites on one or more teeth of the human subject. The term “fluorescence” as used herein, refers to visible light that may be emitted from one or more dental composite substance upon absorption of light energy (i.e., ultraviolet light). The fluorescence emitted is of a longer wavelength than the wavelength of the absorbed light energy. The term “ultraviolet” as used herein, refers to electromagnetic radiation having wavelengths shorter than visible light but longer than X-rays in the range of 100-400 nm. In some embodiments, the ultraviolet light used for the present invention has a spectrum of wavelengths ranging from 100 to about 400 nm, preferably 120 to about 395 nm, more preferably 150 to about 390 nm. In some embodiments, the ultraviolet light has a wavelength of 390 nm. The term “visible light”, as used herein, refers to the visible region of the electromagnetic spectrum having the range of wavelengths that trigger brightness and color perception in humans (i.e., 400-780 nm). In some embodiments, the detecting step of the method may describe the use of the fluorescence or lack of fluorescence of the teeth and/or dental composites due to the excitation wavelengths from ultraviolet light between the specified preferred wavelengths. In some embodiments, the projecting and detecting steps of the method may be performed or repeated multiple times (e.g., 2, 3, 4, 5, 6 times) at a different ultraviolet light wavelength for each repeat. In these embodiments, the method may include a step of adjusting the wavelength of ultraviolet light to be projected on the teeth of the human subject. In these embodiments, the adjusting step may be performed multiple times to set a specific wavelength within the range of about 150 to about 390 nm in each particular instance. Alternatively, in some embodiments, the method may comprise a step of projecting non-ultraviolet light, some exemplary wavelength ranges include 440-490 nm for blue light, 520-570 nm for green light, 625-740 nm for red light and 800-930 nm for infrared light.

In some embodiments, the method may further comprise a step of recording a tooth location for each of the one or more teeth where fluorescence of dental composites was detected. In some embodiments, the projecting is performed with a pen light. In other embodiments, the projecting is performed with a head lamp (e.g., dental overhead light) or a light mounted on a headgear (e.g., dental loupes). The term “dental loupes” refers to special glasses which include a set of magnification lenses that protrude forward from the dentist's or surgeon's face at an acute angle. Loupes tend to be unique to each surgeon as they are opthalmically specific and generally are worn in the same way as a standard pair of glasses, but they have a much higher weight and weight capacity due to the large magnifying lenses used. In some embodiments, the projecting is performed with a light attached to eyeglasses. In some embodiments, the projecting is performed with a dental microscope operating light. In some embodiments, the ultraviolet light has a spectrum of wavelengths ranging from 150-390 nm. In some embodiments, the ultraviolet light has a wavelength of 390 nm. The method may also further include using a camera to perform the detecting step and a computer to perform the recording step.

Another aspect of the disclosure provides a dental inspection tool which comprises a visible light source, an ultraviolet light source, and a switch which switches light projected from the tool between visible light and ultraviolet light. In some embodiments, the tool may be configured as dental loupes, a dental overhead light, a stand lamp, a handheld device, a penlight or a dental microscope. The tool may further comprise a disposable cover while the body of the tool is reusable. In some embodiments, the tool may have a controller to set the light source to emit a specified range of wavelengths. The preferred embodiments of this aspect of the disclosure may comprise a light source, for example, a light emitting diode (LED) (e.g., white LED, blue LED, deep blue LED, UV LED, etc.) or a laser diode, which is different from the light source generated by the normal illumination light.

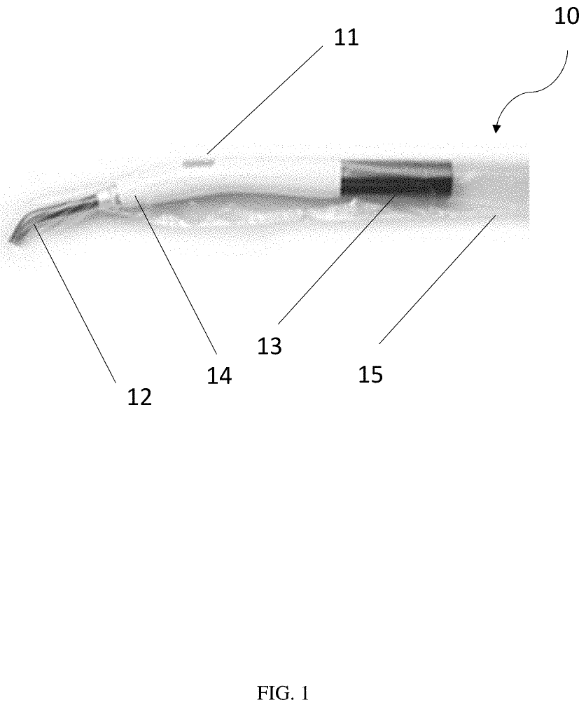

Referring now to FIG. 1, a pen-shaped handheld dental or surgical light tool 10 is shown. The dental tool 10 may include various components and a control panel with a press-button switch 11 on one side thereof together with a detachably secured tube opening 12 for guiding the ultraviolet light to a particular portion of the tooth having embedded dental composites. A power supply 13 (e.g., lithium hydride rechargeable batteries) is electronically connected to a light lamp and shutter and/or wavelength control panel. The light lamp 14 is internally secured to the front portion of the dental tool 10. In some embodiments, the light lamp 14 may emit ultraviolet or blue light. In some embodiments, the light lamp 14 may provide both ultraviolet light and visible light, and the control panel connected to the press-button switch 11 is configured to control the switch between two different light sources. In some embodiments, a removable and disposable cover 15 may be used. The dental tool 10 may also include a window for viewing an optional display of the light setting performed in the control panel. When using the dental tool 10, the main body may be used repeatedly but a new set of disposable cover 15 is used for each patient to avoid transferring disease from one patient to another. The disposable cover 15 may be made of plastic but the plastic material which deliberately includes ultraviolet absorbing materials is not used. In addition, the tube opening 12 of the dental tool 10 is not manufactured with a plastic material having ultraviolet absorbing properties. In preferred embodiment, the disposable cover 15 and/or tube opening 12 are made of one or more materials selected from the group consisting of acrylic, polycarbonate, or other moldable or castable water-clear, transparent, non-brittle plastic material or alloy of plastics.

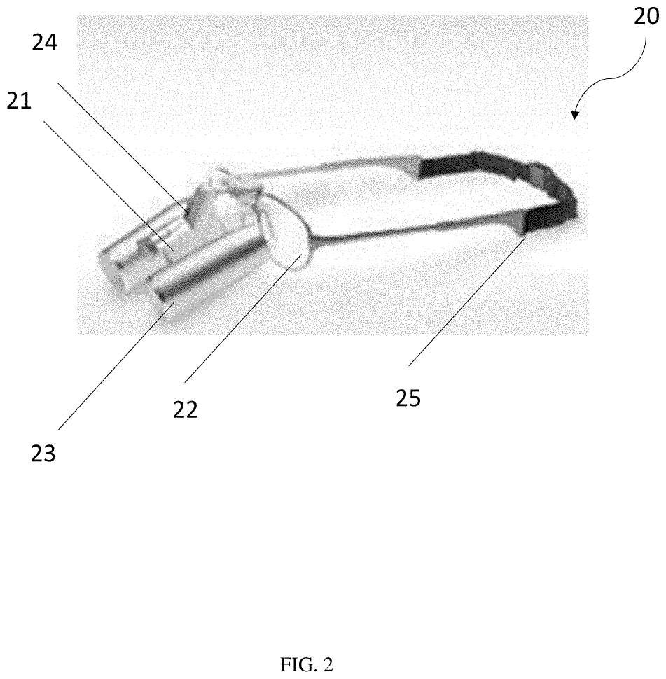

Referring to FIG. 2, a hands-free dental and surgical light system 20, which includes one or more light sources 21 mounted to a pair of optical magnifying loupes 23 and/or safety glasses 22 which can be worn by the dentist or surgeon is shown. In some embodiments, the one or more light sources 21 may include ultraviolet and visible light sources as well as a control system 24 which can turn on and off the light sources 21. The control system may further control each light (i.e., ultraviolet light or visible light) by turning each light source on and off and dim or brighten the light by either manually turning the nob or other methods of adjusting the system such as controlling the system in a hands-free manner. In some embodiments, the ultraviolet or visible light 21 is aligned with the wearer's vision. In some embodiments, the position and alignment of the light 21, loupes 23 and/or glasses 22 may be adjusted by adjusting the strap 25 when the dental tool 20 is worn by the wearer. The glasses portion 22 of the tool may be made from clear pellucid material and has a translucent or transparent region. Alternatively, the translucent region may be transparent but provide with tinting and/or shading and/or opaque in order to protect the wearer's eyes from the reflected ultraviolet light.

In some embodiments, the ultraviolet light source of dental tool 10 and/or 20 may further comprise one or more exciter filters which restrict the ultraviolet radiation primarily to a particular wavelength within the range of about 150 to about 390 nm. The dental tool 10 and/or 20 may further comprise one or more optical filters for receiving the reflected ultraviolet radiation from the teeth and/or dental composites on the teeth to allow only a specific wavelength range of radiation to pass through the filters to the lens for viewing.

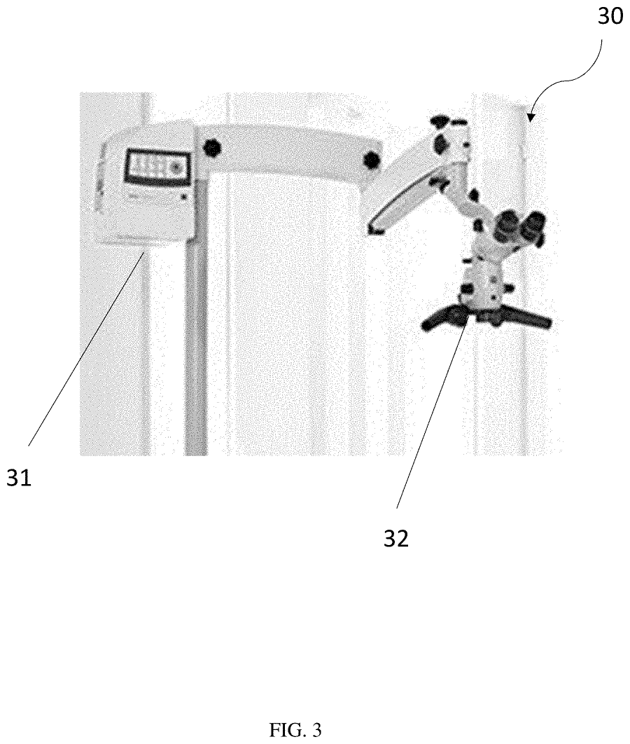

FIG. 3 shows an exemplary dental microscope 30 for observing an ultraviolet fluorescence of dental composite on one or more teeth of a subject. The dental microscope comprises microscopy optics having an objective lens, light emitting sources, one or more filters, an optional camera system, an optional computer system and an optional display system. In preferred embodiments, the dental microscope 30 comprises a control panel 31 for positioning and illuminating ultraviolet light beam through an opening 32 through a filter, wherein the filter only allows a specific wavelength of ultraviolet light to pass. By using a different filter, the reflected emission light from the dental composites upon excitation with the specific wavelength is collected through the same opening 32. The collected light signal is detected, recorded, processed and/or displayed at the control panel 31 or other devices such as additionally connected computers and/or cameras. The collected light can also be viewed by the operator through the dental microscope.

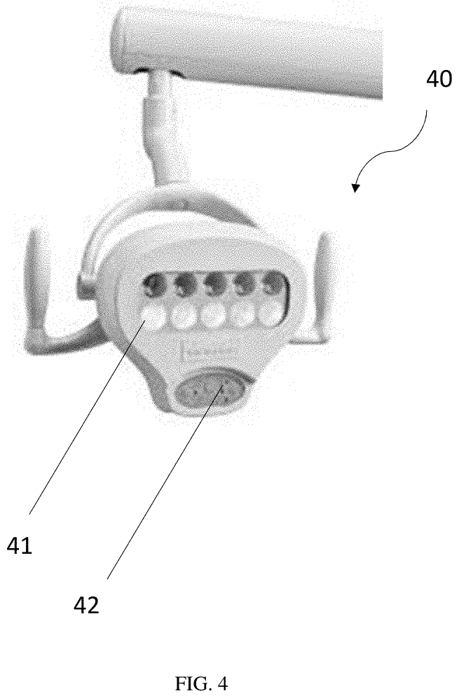

In some embodiments, an ultraviolet and/or visible light source may be mounted on a dental overhead light 40, as shown in FIG. 4. The overhead light dental tool may have multiple openings 41 for ultraviolet and/or visible light set at multiple wavelengths, which may be emitted through the openings individually or simultaneously onto the dental composites on a tooth. The dental overhead light 40 may also comprise a control panel 42 for setting the wavelength of the ultraviolet light source. In preferred embodiments, the dental overhead light 40 comprises a filter in the control panel 42 for setting and illuminating ultraviolet light beam through an opening 41, wherein the filter only allows a specific wavelength of ultraviolet light to pass and selectively filter out the light in outer range of wavelengths.

### Example 1

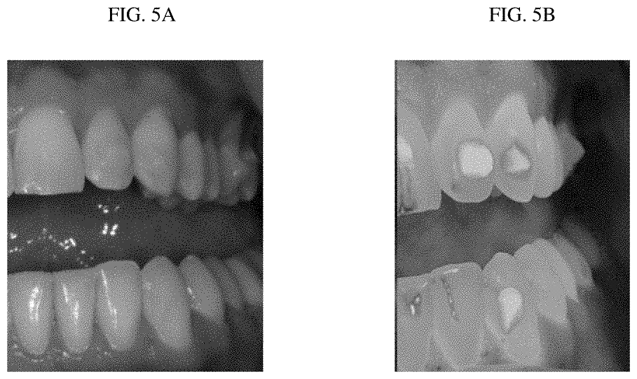

To date, no reliable method for predictably identifying dental composites is available. Dental composites demonstrate a phenomenon called “metamerism” where they appear as different colors under different light sources. Dental composites exposed to UV light will have a fluorescence effect as a result of the metamerism phenomenon (FIGS. 5A-B).

Metamerism, a fundamental psychophysical property of colorimetry, is the phenomenon by which the visual appearance of two-color specimens with different spectral power distributions appears to match. The composite resins may match the color of a natural tooth when observed in daylight or in combination with a conventionally used dental unit lamp but may not match when the light source changes to fluorescence inducing light. When a selected range of ultraviolet wavelengths of light (100 nm-400 nm) were tested, many dental composites were found to fluoresce when exposed to ultraviolet light.

It is to be understood that this invention is not limited to any particular embodiment described herein and may vary. It is also to be understood that the terminology used herein is for the purpose of describing particular embodiments only, and is not intended to be limiting, since the scope of the present invention will be limited only by the appended claims.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

What is claimed is:

1. A method for assessing human teeth, comprising:
projecting an ultraviolet light on the teeth of a human subject; and
detecting fluorescence of dental composites on one or more teeth of the human subject.

2. The method of claim 1 further comprising recording a tooth location for each of the one or more teeth where fluorescence of dental composites was detected.

3. The method of claim 1 wherein projecting is performed with a pen light.

4. The method of claim 1 wherein projecting is performed with a head lamp.

5. The method of claim 1 wherein projecting is performed with a dental microscope operating light.

6. The method of claim 1 wherein projecting is performed with a light mounted on a dental overhead light.

7. The method of claim 1 wherein projecting is performed with a light attached to eyeglasses.

8. The method of claim 1 wherein the ultraviolet light has a spectrum of wavelengths ranging from 100 to 400 nanometers.

9. The method of claim 1 wherein detecting is performed with a camera.

10. The method of claim 2 wherein recording is performed using a computer.

11. A dental inspection tool comprising
a visible light source,
an ultraviolet light source,
a switch which switches light projected from the tool between visible light and ultraviolet light.

12. The tool of claim 11, wherein the tool is a handheld device.

13. The tool of claim 11, wherein the tool is a pen light.

14. The tool of claim 11, wherein the tool is a dental microscope.

15. The tool of claim 11, wherein the tool is attached to dental loupes.

16. The tool of claim 11, wherein the tool is mounted on a dental overhead light.

17. The tool of claim 11, wherein the tool has a controller that sets a wavelength of the ultraviolet light source.

18. The tool of claim 17, wherein the wavelength is selected from a range between about 150 to about 390 nanometers.
